(19) United States
(12) Patent Application Publication
(10) Pub. No.: US 2025/0262438 A1
(43) Pub. Date: Aug. 21, 2025

(54) LOW DUTY CYCLE TEMPORAL INTERFERENCE

(71) Applicant: Weinberg Medical Physics, Inc., North Bethesda, MD (US)

(72) Inventor: Irving N. WEINBERG, Chevy Chase, MD (US)

(21) Appl. No.: 19/057,404

(22) Filed: Feb. 19, 2025

Related U.S. Application Data
(60) Provisional application No. 63/555,465, filed on Feb. 20, 2024.

(51) Int. Cl. A61N 1/36

(52) U.S. Cl. CPC A61N 1/36175; A61N 1/36171

(57) ABSTRACT

Apparatuses and methods for neuromodulation include a plurality of waveform generators configured to generate energetic waveforms and to be arranged around a body part, and a controller configured to control the array to generate low duty cycle energetic waveforms to achieve temporal interference at a selected location in the body part.

### CROSS-REFERENCE AND PRIORITY CLAIM

This application claims priority to U.S. Provisional Patent Application Ser. No. 63/555,465, entitled “LOW DUTY CYCLE TEMPORAL INTERFERENCE” filed Feb. 20, 2024, the entirety of which is incorporated by reference.

### FIELD

An apparatus for and method of neuromodulation are provided. In particular, an apparatus and method of neuromodulation using low duty cycle temporal interference are provided.

### BACKGROUND

It is known that summation of sine waves at different frequencies results in beats, with the frequency of beats being the difference between the summed frequencies. It is also known that when coils produce electromagnetic sine waves that impinge on neural tissues, said neural tissues may be more responsive to the lower, beat frequency than to the higher frequencies of the electromagnetic sine waves, as taught in 2022 by Adam Khalifa et al and then published in the Journal of Neural Engineering publication entitled “Magnetic Temporal Interference for Noninvasive Focal Brain Stimulation”, visible at doi: 10.1088/1741-2552/acb015. As taught by Khalifa, an advantage of magnetic temporal interference (MTI) is that when the coils are placed at different locations, then the amplitude of the perceived electromagnetic pulse at the beat frequency is higher at a position between the coils than near each coil. This principle is useful when neuromodulation of deep tissues (far from the coils) is desired, without over-stimulation of superficial tissues (which are near the coils). The MTI phenomenon further takes advantage of a physiological principle in which the neurological response to electromagnetic fields depends on frequency (with higher threshold for response at higher frequency).

A technical challenge to implementing magnetic temporal interference (MTI) as originally taught by Khalifa is that the electromagnetic sine waves are generated at 100% duty cycles, which requires considerable power and cooling (as compared to generation of electromagnetic waves at lower duty cycles).

### SUMMARY

Disclosed embodiments provide a means of achieving MTI using electromagnetic waves (or other energetic waves, for example sound waves) generated at lower duty cycles, thereby permitting use in neuromodulation devices with low power capabilities and reducing or eliminating the need for cooling the equipment that generates the electromagnetic waves.

An apparatus for (and method of) neuromodulation is provided, in which MTI is applied using electromagnetic waves with low duty cycles.

### BRIEF DESCRIPTION OF THE FIGURES

FIG. 1 shows an embodiment of an apparatus for MTI; and

FIG. 2 illustrates an embodiment of a method in which MTI is used to achieve a physiological result in a human or non-human animal.

### DETAILED DESCRIPTION

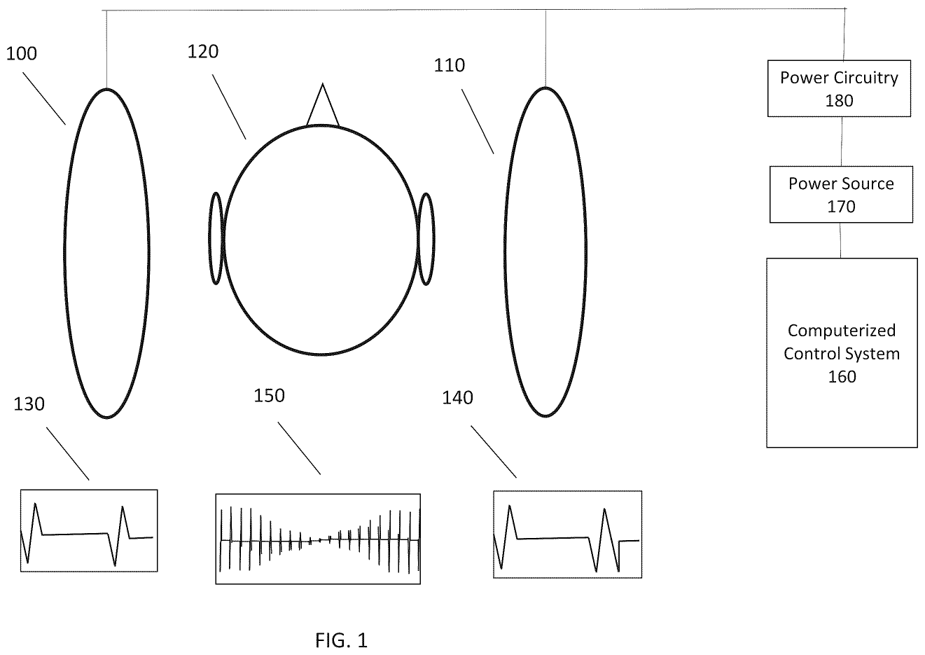

As illustrated in FIG. 1, the apparatus may have an array of coils 100 and 110 for generating electromagnetic waves 130 and 140 (respectively) that may be placed at different locations around a human or non-human animal subject's body part (shown as a human head 120). Although only two coils are shown, it is understood that many such coils could be used. Coils may be connected via wires to one or more control systems 160 and power sources 170 energizing the coils.

Although coils are shown as waveform generators, it is understood that other means of generating electromagnetic waves could be used. For example, wave generators may be moving permanent magnetized rods. A representative electromagnetic wave form generated by coil 100 is shown as 130. For the purposes of this disclosure, the terms electromagnetic and magnetic are used interchangeably, although it is understood that every electromagnetic wave has a magnetic component and vice versa. The electromagnetic wave form is shown in 130, as a trace of magnetic field versus time, as a single magnetic sine wave followed by a period of no magnetic field, in which the overall duty cycle of the electromagnetic wave is low. Similarly, a representative electromagnetic wave form generated by coil 110 is shown as 140, with slightly different frequency as compared to 130. A representation of the sum of the two magnetic fields at a location between the two coils is shown as 150, where the time scale has been reduced in size by about a factor of 10 so that the beating phenomenon can be observed. As can be seen in FIG. 1, there is beating of the magnetic fields at this location, with the beat having a lower frequency than the sine waves used to generate the beating frequency, and at much lower duty cycle than if the electromagnetic sine waves had been continuous.

The apparatus contains a computerized control system 160, power source 170 (e.g., capacitors and/or batteries), power circuitry 180 (e.g., transistors, diodes), which together act to controlling the currents and voltages sent to the means (for example, coils) of generating electromagnetic waves 100 and 110, said means being in an array at least partly surrounding a body part 120. The apparatus is described herein as a “waveform generator” or “generator” or “generating means”. It is understood that the array may contain many such generating means, and that the generating means may include a coil and/or magnetizable material. It is understood that a similar low-duty-cycle principle may be applied to other energetic waves, for example, acoustic waves.

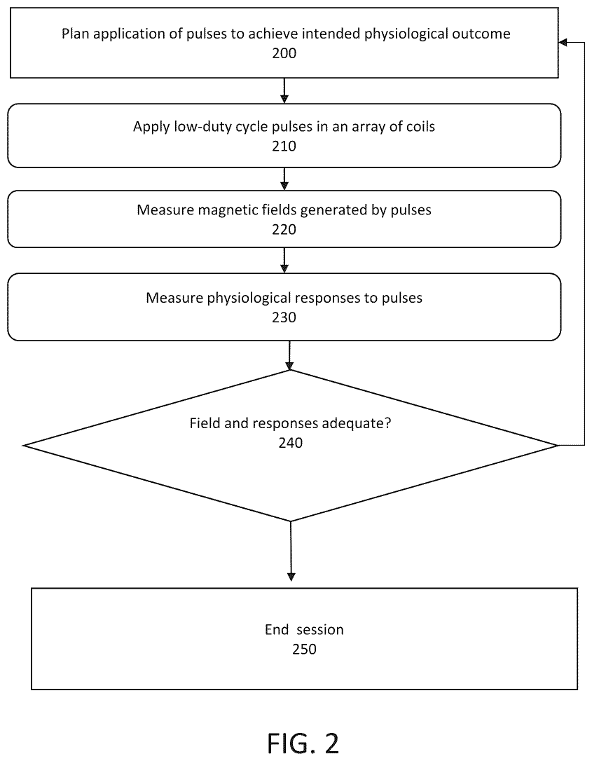

FIG. 2 is a flowchart of a method of MTI for neuromodulation. In operation 200, a plan is devised with the aid of a computer to apply electromagnetic fields to one or more pre-selected regions in a body part. In operation 210, multiple low duty cycle magnetic pulses are applied to the body part from an array of at least two coils or other magnetic field generators, such that the degree of neurological responses produced at specific locations within the body part are preferentially modulated in response to the predetermined beating frequencies and magnitudes at these locations. In optional operation 220, the properties of the electromagnetic fields (for example, magnitude and direction) at various locations within the body part may be measured, for example using magnetic resonance imaging (MRI) as taught by D. E. Bohning et al in the 1997 Neuroreport article entitled “Mapping transcranial magnetic stimulation (TMS) fields in vivo with MRI”. In optional operation 230, the physiological response of neurons may be measured, for example with functional MRI or electroencephalography (EEG) or another method. In optional operation 240, the measurements of the electromagnetic fields and or the physiological responses obtained with operations 220 and 230, respectively, may be compared with the plan of operation 200, and if the desired outcome has been achieved, the session may be terminated as in operation 250. For example, a desirable physiological response may be the production of a current in a neuronal tract as measured with current imaging, using the methods of B. Sveinsson et al, in the J Neural Eng 2020 publication entitled “Detection of nanotesla AC magnetic fields using steady-state SIRS and ultra-low field MRI”, with DOI 10.1088/1741-2552/ab87fe.

The results shown in FIG. 1 were obtained with a SPICE (Simulation Program with Integrated Circuit Emphasis) simulation involving generation of currents summed with an ideal operational amplifier circuit modeling neural response. The use of summing operational amplifiers to model neuronal response is known, having been taught by F. Sarwar, S. Iqbal, and M. W. Hussain in the 2016 Z. Naturforsch article entitled “Linear and Nonlinear Electrical Models of Neurons for Hopfield Neural Network”. The parameters of the two waveforms 130 and 140 were as follows: For waveform 130, a sine wave was generated with frequency 100,000 Hz, which was gated by a switch that allowed current to flow for 8.3 microseconds, with a period of 50 microseconds. For waveform 140, a sine wave was generated with frequency 101,000 Hz, which was gated by a switch that allowed current to flow for 8.3 microseconds, with a period of 50 microseconds.

The disclosed method for neuromodulation describes an embodiment of operation of the apparatus to achieve interference effect with low duty cycles, thereby reducing the average power of the device and the need to cool the coils to dissipate said power. For the purposes of this disclosure, the low duty cycles of the apparatus operated with the method may be less than 1%, less than 5%, less than 10%, or less than 25%. The MTI effect is achieved according to the disclosed embodiments with low duty cycles, reduces the average power of the device and the need to cool the coils (and other components of the power chain) to dissipate said power.

It is understood that the frequency and magnitude of energetic waveforms generated by at least one of the coils may be high enough that there is little or no physiological response (i.e., sensation) near the coils (e.g., within 5-cm of the coils), as taught by I. N. Weinberg et al in the 2012 Medical Physics publication entitled “Increasing the oscillation frequency of strong magnetic fields above 101 kHz significantly raises peripheral nerve excitation thresholds”. The high threshold frequency may be above 10 kHz, above 20 kHz, above 50 kHz, above 101 kHz, or above 200 kHz. It is understood that at a selected location in the body part, the beat frequency and magnitude of the interfering waveforms may be above the threshold for sensation. An example of a commonly-used threshold for low-frequency sensation (e.g., <10 Hz) due to electromagnetic fields is 100 volts per meter.

It is understood that a multiplicity of generators may be used to achieve a tomographic effect within tissue at a selected location in a body part. It is understood that the generators may be in modules that may achieve both imaging and neuromodulation effects, as taught by I. N. Weinberg in US20210402200.

Although the waveforms shown in FIG. 1 are represented as sine waves followed by a rest period, similar MTI results may be obtained with other low-duty cycle waveforms, for example asymmetric rising and falling magnetic fields followed by a rest period (said rest period involving a magnetic field of lower or no magnitude).

Those skilled in the art will recognize, upon consideration of the above teachings, that the above exemplary embodiments and the control systems may be based upon use of one or more programmed processors programmed with a suitable computer program. However, the disclosed embodiments could be implemented using hardware component equivalents such as special purpose hardware and/or dedicated processors. Similarly, general purpose computers, microprocessor-based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out the above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While various exemplary embodiments have been described above, it should be understood that they have been presented by way of example only, and not limitation. Thus, the breadth and scope of the present invention should not be limited by any of the above-described exemplary embodiments, but should instead be defined only in accordance with the following claims and their equivalents.

1. An apparatus for neuromodulation comprising:
a plurality of waveform generators configured to generate energetic waveforms and to be arranged around a body part, and
a controller configured to control the array to generate low duty cycle energetic waveforms to achieve temporal interference at a pre-selected location in the body part.

2. The apparatus of claim 1, wherein the energetic waveforms are electromagnetic.

3. The apparatus of claim 1, wherein the energetic waveforms are acoustic.

4. The apparatus of claim 1, wherein a duty cycle of the low duty-cycle energetic waveforms is less than 25%.

5. The apparatus of claim 1, wherein a duty cycle of the low duty-cycle energetic waveforms is less than 1%.

6. The apparatus of claim 1, wherein a frequency of at least one of the plurality of waveform generators is above the threshold for sensation.

7. The apparatus of claim 6, wherein the frequency is above 10 KHz.

8. The apparatus of claim 6, wherein the frequency is above 101 kHz.

9. A method for neuromodulation comprising:
arranging a plurality of waveform generators around a body part;
applying low duty-cycle energetic waveforms to achieve temporal interference in a pre-selected location within the body part.

10. The method of claim 9, wherein the energetic waveforms are electromagnetic.

11. The method of claim 9, wherein the energetic waveforms are acoustic.

12. The method of claim 9, wherein a duty cycle of the low duty-cycle energetic waveforms is less than 25%.

13. The apparatus of claim 9, wherein a duty cycle of the low duty cycle energetic waveforms is less than 1%.

14. The method of claim 9, wherein a frequency and magnitude of at the least one waveform generator is above a threshold for sensation at the pre-selected location within the body part and a beat frequency and magnitude is above said threshold.

15. That the method of claim 14, wherein the frequency is above 10 kHz.

16. The method of claim 14, wherein the frequency is above 101 kHz.
